(19) United States
(12) Patent Application Publication
(10) Pub. No.: US 2014/0220607 A1
(43) Pub. Date: Aug. 7, 2014

(54) SIGNAL AMPLIFICATION FOR IMMUNOASSAYS BY USE OF AVIDIN-BIOTIN LINKAGES

(71) Applicant: Bio-Rad Laboratories, Inc., Hercules, CA (US)

(72) Inventors: Woei Tan, Hercules, CA (US); Nobuo Monji, Seattle, WA (US); Vinita Gupta, Danville, CA (US); Candice Cox, El Cerrito, CA (US)

(73) Assignee: Bio-Rad Laboratories, Inc., Hercules, CA (US)

(21) Appl. No.: 14/165,205

(22) Filed: Jan. 27, 2014

Related U.S. Application Data
(62) Division of application No. 13/411,846, filed on Mar. 5, 2012, now abandoned.
(60) Provisional application No. 61/449,463, filed on Mar. 4, 2011.

(51) Int. Cl. G01N 33/543

(52) U.S. Cl. CPC G01N 33/54306
USPC 435/7.94; 436/501

(57) ABSTRACT

In sandwich-type immunoassays that capture a protein analyte between a capture antibody, typically bound to a solid phase, and a detection antibody that is coupled to a reporter group, the number of reporter groups associated with each molecule of analyte is increased by a variety of methods that utilize avidin-biotin-type binding in conjunction with such features as immunological binding to the reporter group on the detection antibody or multiple biotin-avidin-type binding sites.

### CROSS-REFERENCE TO RELATED APPLICATION

This application is a Divisional application of U.S. application Ser. No. 13/411,846, filed Mar. 5, 2012, which claims the benefit of U.S. Provisional Patent Application No. 61/449,463, filed Mar. 4, 2011. The contents of the aforementioned applications of which are incorporated herein by reference in their entirety.

### BACKGROUND OF THE INVENTION

Most immunoassays for the detection of proteins follow the well-known “sandwich” format in which the analyte protein is first bound to a capture antibody attached to a solid phase, then bound to a detection antibody which is labeled with a reporter group such as a fluorophore, an enzyme, or another protein to ultimately obtain a detectable signal. Examples of a non-enzymatic binding member that can be used as the reporter group are biotin, avidin, or streptavidin, in which cases the binding of the reporter group-bearing detection antibody is followed by binding of the reporter group to its binding partner in biotin-avidin binding, with the binding partner bearing an enzyme or fluorophore. In general, the magnitude of the signal is determined by the reporter group or, when two or more reporter groups are attached to the detection antibody, by the number of reporter groups, and not amplified further.

### SUMMARY OF THE DISCLOSURE

It has now been discovered that the number of reporter groups can be increased beyond those that are attached to the detection antibody, by a variety of methods, including those that take further advantage of biotin-avidin-type binding as well as those that involve immunological binding to the reporter group on the detection antibody, and those that involve species with multiple biotin-avidin-type binding sites, such as polybiotin.

### BRIEF DESCRIPTION OF THE DRAWINGS

FIG. 1 is a diagrammatic representation of a protocol which is an example of one method in accordance with the present invention.

FIG. 2 is a diagrammatic representation of a protocol which is an example of a second method in accordance with the present invention.

FIG. 3 is a diagrammatic representation of another protocol which is an example of the same method as the example of FIG. 2.

FIG. 4 is a diagrammatic representation of still another protocol which is an example of the same method as the example of FIG. 2.

FIG. 5 is a diagrammatic representation of a protocol which is an example of a third method in accordance with the present invention.

FIG. 6 a diagrammatic representation of another protocol which is an example of the same method as the example of FIG. 5.

FIG. 7 is a diagrammatic representation of a protocol which is an example of a fourth method in accordance with the present invention.

### DETAILED DESCRIPTION OF SELECTED EMBODIMENTS

In each of these methods, the sample to be assayed for the presence of the analyte of interest is first incubated with an immunological binding member that is bonded to a solid support, the binding member being one that has selective binding affinity for the analyte. This immobilizes the analyte on the support, where successive binding reactions are performed to bind reporter groups to the analyte through one or a succession of further binding reactions.

In one of the methods, the solid support, to which the analyte if present in the sample is now bound, is incubated with a second immunological binding member that has selective binding affinity for the analyte, to form a complex with the analyte and the first immunological binding member in the “sandwich” manner. The second immunological binding member is one that is labeled with two or more copies of a reporter group, and hence the complex formed in this step contains two or more reporter groups for each molecule of analyte. Once this “sandwich” complex is formed, the solid phase is incubated with a third immunological binding member that has selective binding affinity for the reporter group, and thereby extends the complex on the solid phase further by adding at least one copy of the third immunological binding member for each copy of the reporter group already included in the complex. In some procedures, multiple (two or more) copies of the third immunological binding member will be attached for each copy of the reporter group. The third immunological binding member also has the distinction of being coupled to an affinity-type binding member within the avidin-biotin family. The members of this family include avidin, streptavidin, biotin, polybiotin, and any other species that engage in an avidin-biotin interaction with another member of the family. The label can thus, for example, be biotin, which will form a complex in the succeeding binding reaction with either avidin or streptavidin. In certain embodiments of this method, the third immunological binding member will be coupled either with two or more copies of the affinity-type binding member, or with an affinity-type binding member that itself will bind to two or more counterparts within the avidin-biotin family. Polybiotin for example will bind to multiple copies of avidin or streptavidin, and avidin and streptavidin will each bind with multiple copies of biotin. Preferably, however, the affinity-type binding member coupled to the third immunological binding member is biotin, and two or more copies of biotin will be coupled to each copy of the third immunological binding member.

The final binding reaction in this first method is performed by incubating the solid phase with an affinity-type binding member of the avidin-biotin family that engages in an avidin-biotin interaction with the binding member added to the complex in the preceding step. Each copy of this second affinity-type binding member will be labeled with a reporter group, preferably the same reporter group included in the first incubation, prior to this final binding reaction. The resulting complex on the solid support will thus contain multiple copies of the reporter group, including those from the first incubation plus those from the second incubation, for each molecule of analyte.

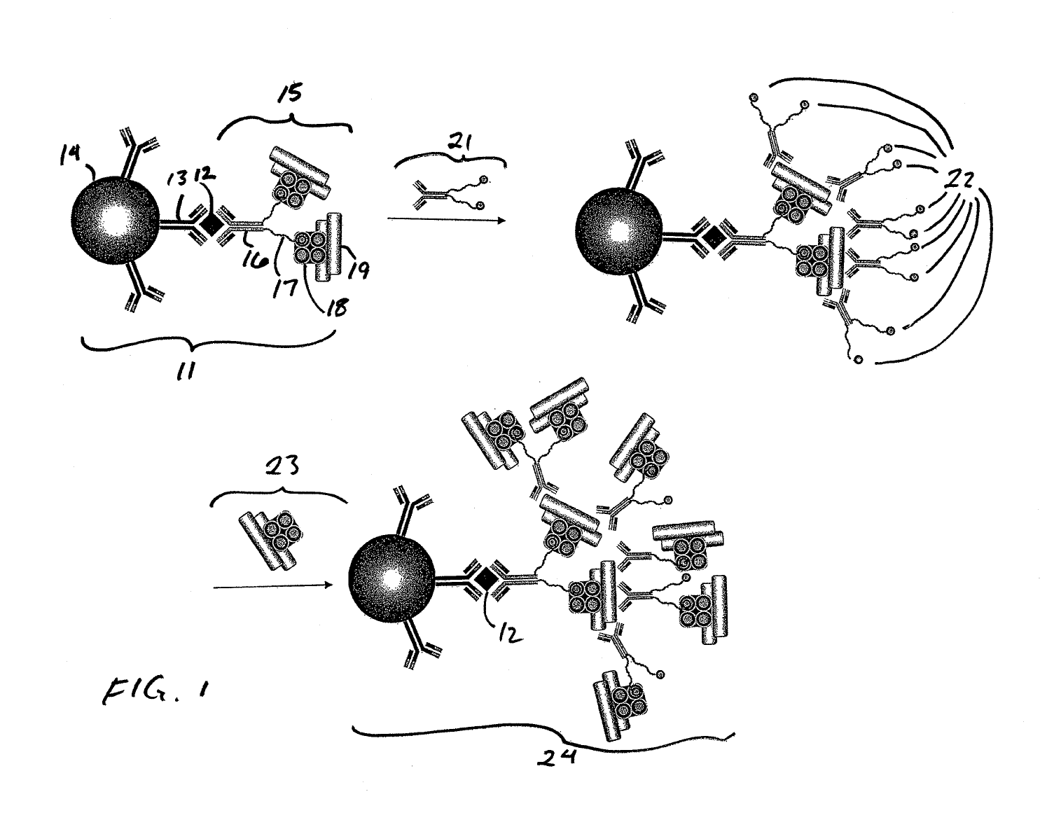

FIG. 1 illustrates this first method. Complex 11 is the initial sandwich complex formed by first incubating the analyte 12 with a capture antibody 13 (the first immunological binding member) that is coupled to a solid support 14, and then incubating the solid support (together with its bound analyte) with a conjugate 15 of a second antibody 16 (the second immunological binding member), biotin 17, streptavidin 18, and a reporter group 19 which in this example is phycoerythrin. Once the sandwich complex 11 is formed, it is incubated with biotin-conjugated anti-phycoerythrin antibody 21 (the third immunological binding member) which adds multiple biotin sites 22 to the complex. The final incubation is with phycoerythrin-labeled streptavidin 23, to produce a final complex 24 that contains a multitude of phycoerythrin groups joined to the single analyte 12 molecule through the various affinity-type and immunological linkages. In this case, the second antibody 16 is coupled to two biotin moieties, each of which bears a separate phycoerythrin group through the avidin-biotin linkage, and each biotin-conjugated anti-phycoerythrin antibody 21 bears two phycoerythrin labels. The result in the final complex is a minimum of six phycoerythrin labels per analyte molecule, the representation in the Figure showing eight.

In a second general method in accordance with this invention, the solid support to which the analyte has become bound through the first immunological binding member in the initial analyte immobilization step is incubated with a second immunological binding member that has selective binding affinity for the analyte, to form, as in the first method, a complex with the analyte and the first immunological binding member in the “sandwich” manner. The second immunological binding member is one that is labeled with two or more copies of a first affinity-type binding member of the avidin-biotin family, so that the resulting complex contains two or more copies of the affinity-type binding member for each molecule of analyte. A counterpart affinity-type binding member and a reporter group are added to the complex in one of two ways: (1) the first affinity-type binding member is already (prior to the incubation) bound to a counterpart affinity-type binding member that is labeled with a reporter group, or (2) the labeled counterpart affinity-type binding member is bound after the incubation by a succeeding incubation. In either case, the resulting complex on the solid phase contains two or more reporter groups, each bound to the single analyte molecule through an avidin-biotin complex. The solid phase is then incubated with a member of the avidin-biotin family that is a counterpart to the affinity binding member utilized in the preceding incubation, this latest member either being labeled with a reporter group or being coupled to a immunological binding member. In the latter case, the immunological binding member will be coupled to two or more affinity binding members to serve as a bridge for further attachment of a reporter group. The affinity binding members in the final complex will thus form a linkage that includes two or more biotin moieties bound to a single avidin (or streptavidin) moiety, and a relatively large number of reporter groups bound to each molecule of analyte.

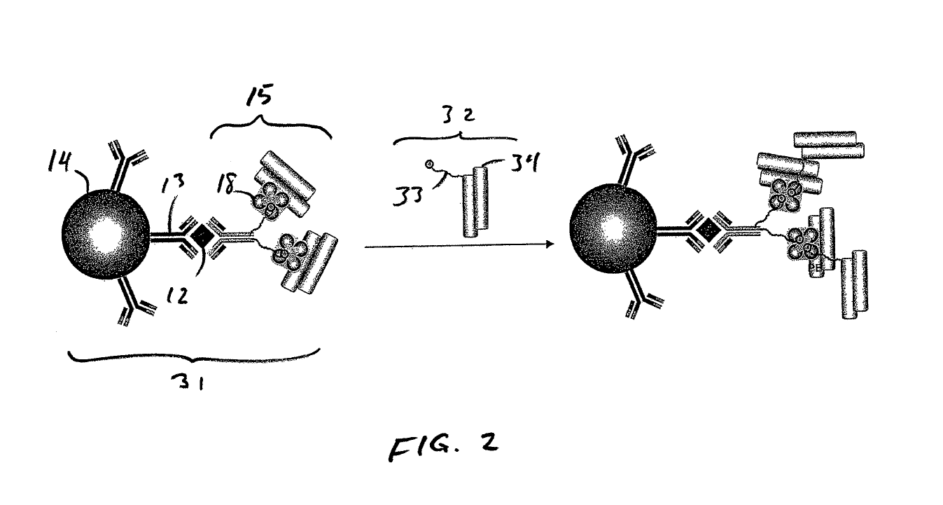

FIGS. 2 through 4 are illustrations of protocols according to the second method. In the protocol of FIG. 2, complex 31 is the initial sandwich complex formed by first incubating the analyte 12 with a capture antibody 13 (the first immunological binding member) that is coupled to a solid support 14, and then incubating the solid support (together with its bound analyte) with the same conjugate 15 used in the example of FIG. 1. The subsequent binding reaction in this example is between the sandwich complex 31 and a conjugate 32 of biotin 33 and phycoerythrin 34. The biotin 33 portion of the conjugate forms a bond with an unoccupied binding site on the streptavidin moiety 18, thereby adding more copies of the phycoerythrin label to the streptavidin moiety. The result is a final complex with a total number of phycoerythrin moieties that equals the sum of the phycoerythrin moieties used in the last two incubations. In this case, the second antibody 16 is coupled to two biotin moieties, each of which bears its own separate phycoerythrin label through the avidin-biotin linkage, and the final complex contains a total of four phycoerythrin labels.

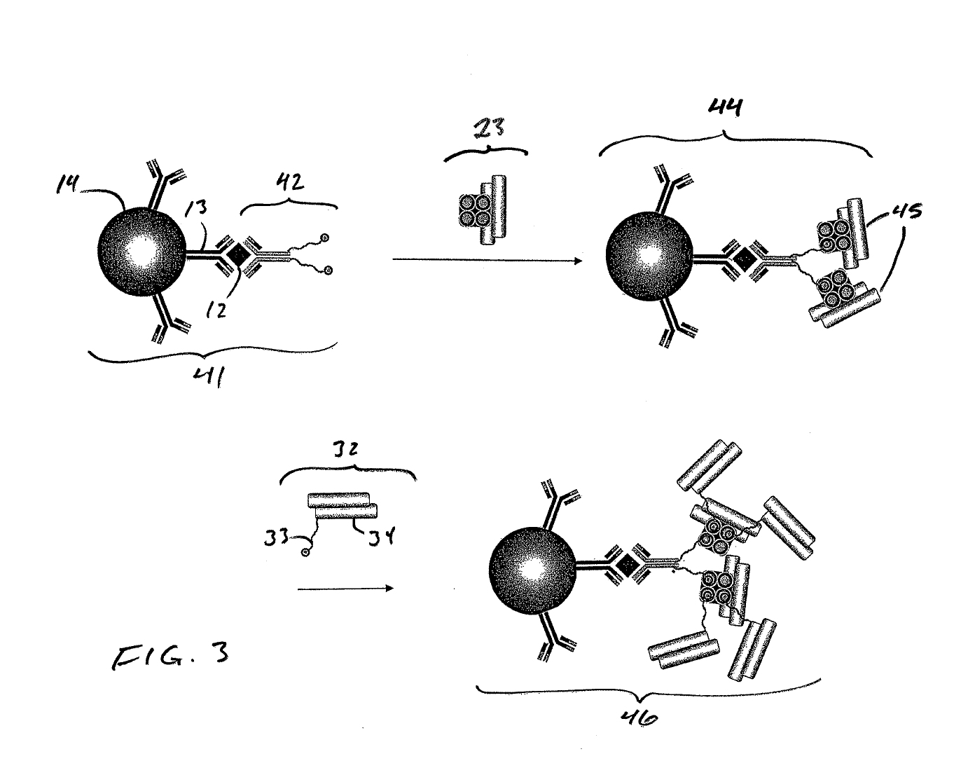

In the protocol of FIG. 3, complex 41 is the initial sandwich complex formed by first incubating the analyte 12 with a capture antibody 13 (the first immunological binding member) that is coupled to a solid support 14, and then incubating the solid support (together with its bound analyte) with biotin-conjugated antibody 42 (the third immunological binding member) that has specific binding affinity for the analyte. This is followed by incubation with a conjugate 23 of phycoerythrin and streptavidin to form a complex 44 that includes a separate phycoerythrin label 45 for each biotin moiety included in the biotin-conjugated antibody 42. The final binding reaction in this example is between the extended complex 44 and a conjugate 32 of biotin 33 and phycoerythrin 34. The biotin 33 portion of the conjugate forms a bond with an unoccupied binding site on the avidin moiety of the phycoerythrin-avidin complex 23, thereby adding more copies of the phycoerythrin label to the streptavidin moiety. The result here again is a final complex 46 with a total number of phycoerythrin moieties that equals the sum of the phycoerythrin moieties introduced in the last two incubations. In this case, the second antibody 42 is coupled to two biotin moieties, each of which bears its own separate phycoerythrin label in the extended complex 44 through an avidin-biotin linkage, and a sufficient number of phycoerythrin-biotin conjugates 32 are employed in the final incubation to result in two additional biotin moieties being added to each avidin moiety. The final complex contains a total of six phycoerythrin labels.

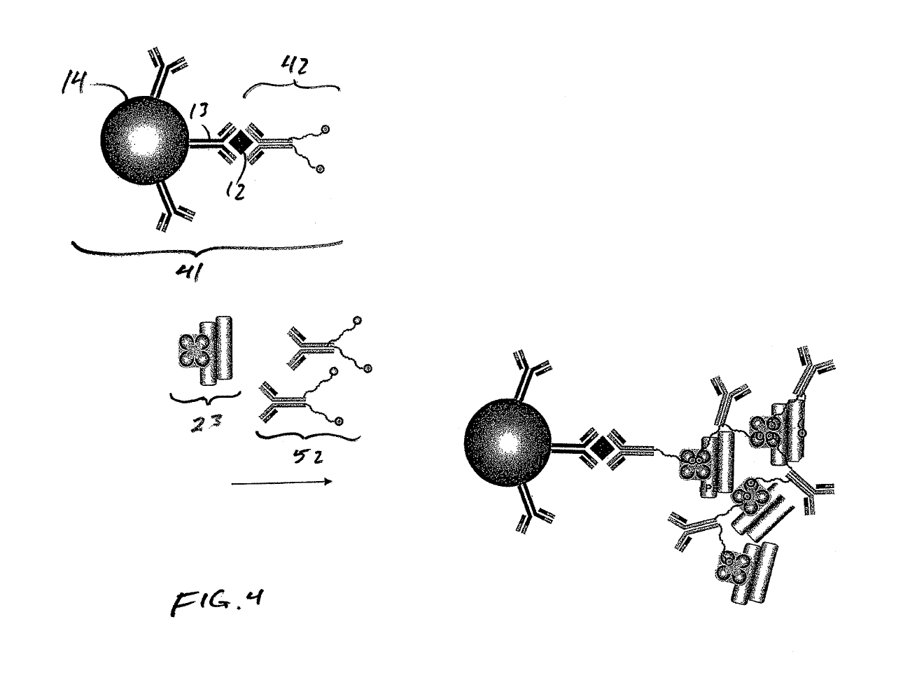

In the protocol of FIG. 4, the initial sandwich complex 41 is the same as that of the protocol of FIG. 3, but the sandwich complex once formed is then incubated with excess quantities of both phycoerythrin-labeled streptavidin 23 and biotinylated antibody 52 in which each antibody is labeled with at least two biotin moieties. In this incubation, a portion of the phycoerythrin-labeled streptavidin 23 will bind to the biotin moieties on the biotin-conjugated antibody 42 that forms the outer part of the initial sandwich complex 41, while the biotinylated antibody 52 serves as a bridge between the phycoerythrin-labeled streptavidin 23 that is part of the initial sandwich complex and additional phycoerythrin-labeled streptavidin 23 included in the second incubation. The antibody-binding function itself of the antibody is thus not utilized. The result is a final complex 53 that contains multiple labels for each analyte molecule.

In a third general method in accordance with this invention, the solid support to which the analyte has become bound through the first immunological binding member in the initial analyte immobilization step is incubated with a biotin multimer in one of the succeeding incubation steps, and several or most of the biotin sites on the multimer will ultimately be bound to avidin or streptavidin, labeled with the reporter group. The final complex thus contains multiple copies of the reporter group bound to each solid-phase-bound analyte molecule through avidin-biotin-type linkages on the biotin multimer. Examples of the biotin multimer are biotin dendrimers and other polybiotins. One implementation of this method is to use a biotin multimer that is coupled directly to the second immunological binding member that completes the “sandwich” complex. The biotin multimer in this case will thus be part of the first incubation after the analyte is initially captured by the solid phase. Another implementation is use an immunological binding member conjugated to avidin or streptavidin as the immunological binding member that completes the “sandwich” complex, and to incubate the avidin or streptavidin-labeled sandwich complex with a complex that consists of a biotin multimer bound to two or more avidin or streptavidin moieties, each of which is labeled with a reporter group.

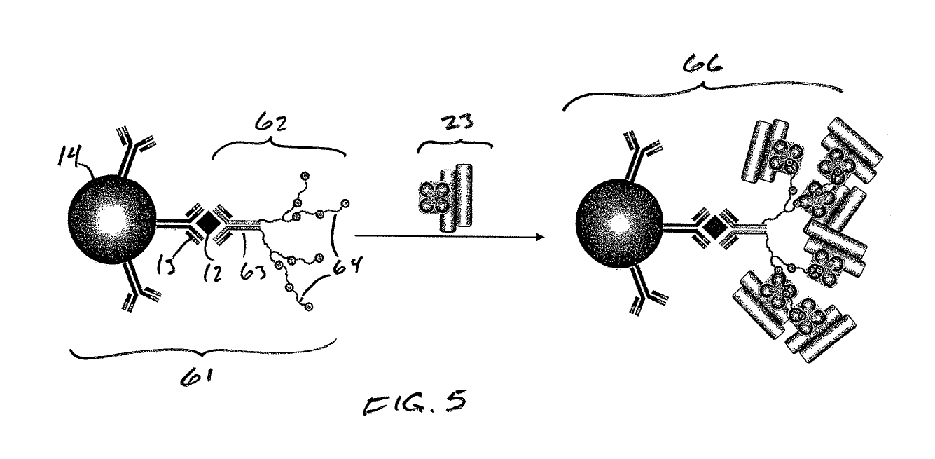

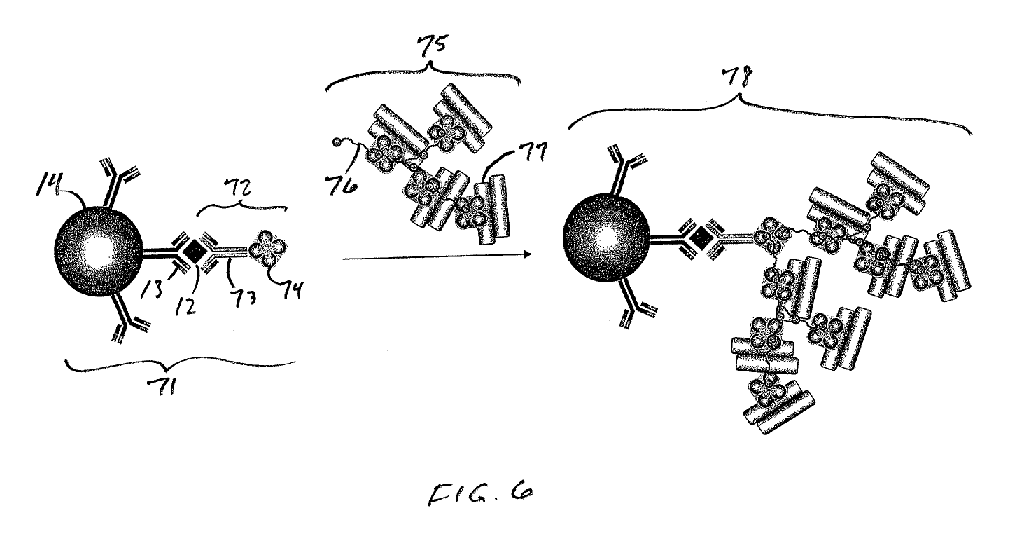

FIGS. 5 and 6 are illustrations of protocols according to the third method. In the protocol of FIG. 5, the initial complex 61 is formed by first incubating the analyte 12 with a capture antibody 13 (the first immunological binding member) that is coupled to a solid support 14, and then incubating the solid support (together with its bound analyte) with a conjugate 62 of an antibody 63 with specific binding affinity for the analyte and at least one biotin multimer 64. In the example shown, two biotin pentamers are coupled to a single antibody molecule. In the succeeding step, the complex is incubated with phycoerythrin-labeled streptavidin 23 in a quantity sufficient to cause avidin-biotin-type binding to occur at two or more biotin sites on each multimer. The number of labels on the final complex 66 is equal to the number of biotin sites on the multimer(s) that have become bound in the last incubation; in this case, the total shown is six phycoerythrin moieties for each molecule of analyte.

In the protocol of FIG. 6, the initial sandwich complex 71 is formed by first incubating the analyte 12 with a capture antibody 13 (the first immunological binding member) that is coupled to a solid support 14, as in all of the protocols described above, but then incubating the solid support and bound analyte with a conjugate 72 of an antibody 73 with specific binding affinity for the analyte and streptavidin 74. The biotin multimer is utilized in the succeeding incubation, in which the initial sandwich complex 71 is incubated with a complex 75 of the biotin multimer 76 and phycoerythrin-labeled streptavidin 77. This incubation results in a final complex 78 that contains a multitude of phycoerythrin labels joined to each analyte molecule through the biotin multimer, which in the example shown is a dendrimer.

In a fourth general method in accordance with the invention, the solid support to which the analyte has become bound through the first immunological binding member in the initial analyte immobilization step is incubated with a second immunological binding member that has selective binding affinity for the analyte, to form a complex with the analyte and the first immunological binding member in the “sandwich” manner. The second immunological binding member is one that is labeled with a single copy of a reporter group. Once this “sandwich” complex is formed, the solid phase is incubated with multiple copies of a third immunological binding member that has selective binding affinity for the reporter group, and thereby extends the complex on the solid phase further by adding at least multiple copies of the third immunological binding member for each copy of the reporter group already included in the complex, the third immunological binding member itself being coupled to biotin. The solid phase is then incubated with avidin or streptavidin labeled with a reporter group to produce a final complex that contains multiple copies of the reporter group for each molecule of analyte.

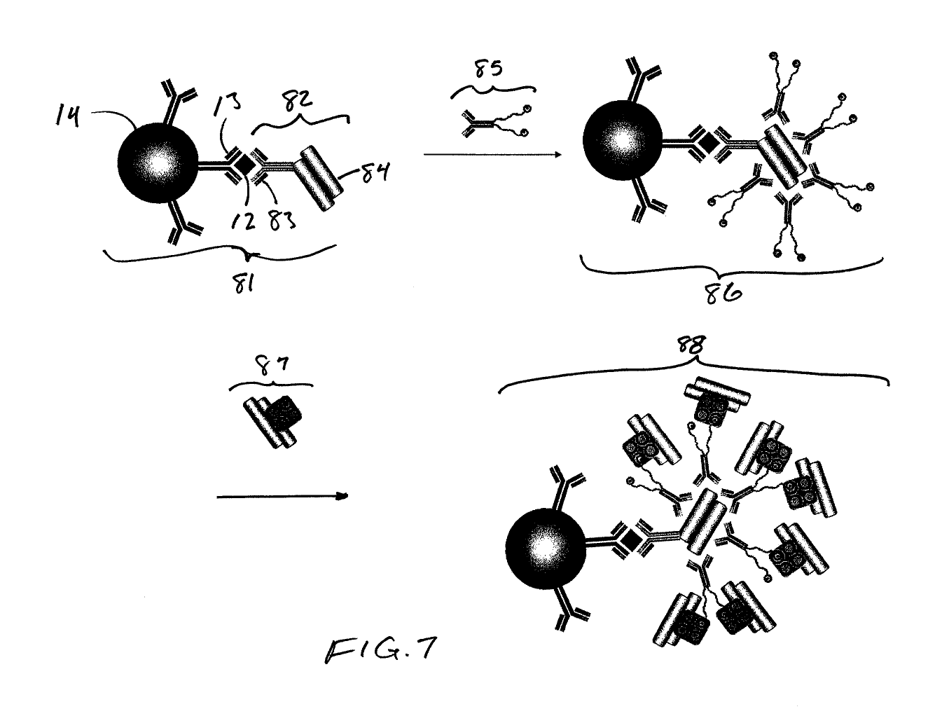

This method is illustrated in FIG. 7, where the initial sandwich complex 81 is formed by first incubating the analyte 12 with a capture antibody 13 (the first immunological binding member) that is coupled to a solid support 14, as in the protocols described above, but then incubating the solid support and bound analyte with a conjugate 82 of a antibody 83 that has specific binding affinity for the analyte and is labeled with phycoerythrin 84. The resulting complex is then incubated with an excess amount of biotin-coupled antibody 85 that has specific affinity for phycoerythrin 84, resulting in a complex 86 that contains multiple copies of the biotin-coupled antibody 85 becoming bound to each particle of the solid phase. This complex 86 is then incubated with phycoerythrin-labeled streptavidin 87 to form the final complex 88 which contains but a single molecule of the analyte 12 and multiple copies of phycoerythrin.

While phycoerythrin is the reporter group in the above examples, any reporter group known for use in immunoassays can be used. Other fluorophores include acridine, acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate, N-(4-anilino-1-naphthyl)maleimide, anthranilamide, BODIPY, coumarins, cyanine dyes, cyanosine, 4′,6-diaminidino-2-phenylindole (DAPI), 5′,5″-dibromopyrogallol-sulfonaphthalein, 5-[dimethylamino]naphthalene-1-sulfonyl chloride (DNS), 4-(4′-dimethylaminophenylazo)benzoic acid (DABCYL), 4-dimethylaminophenylazophenyl-4′-isothiocyanate (DABITC), eosin, 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), 6-carboxy-X-rhodamine (ROX), rhodamine B, and N,N,N′,N′-tetramethyl-6-carboxyrhodamine (TAMRA). Other reporter groups are radioactive labels and enzymes. Examples of enzymes are horseradish peroxidase, chloramphenicol acetyl transferase, β-galactosidase, alkaline phosphatase, and luciferase. The solid support can be any material that is inert to the reactions in the assay and that can be separated from the liquids in the assay. Beads, microbeads, are common examples, although flat solid surfaces or the walls of receptacles can also be used. Finally, while antibodies are used in the examples as the immunological binding members, antibody fragments can also be used. Other substitutions and variations in the various features and components set forth above will be apparent to those skilled in the art.

In the claims appended hereto, the term “a” or “an” is intended to mean “one or more.” The term “comprise” and variations thereof such as “comprises” and “comprising,” when preceding the recitation of a step or an element, are intended to mean that the addition of further steps or elements is optional and not excluded. All patents, patent applications, and other published reference materials cited in this specification are hereby incorporated herein by reference in their entirety. Any discrepancy between any reference material cited herein or any prior art in general and an explicit teaching of this specification is intended to be resolved in favor of the teaching in this specification. This includes any discrepancy between an art-understood definition of a word or phrase and a definition explicitly provided in this specification of the same word or phrase.

1-30. (canceled)

31. A method for detecting an analyte in a sample, said method comprising:
(a) forming an analyte sandwich between the analyte, a first immunological reagent bound to a solid support, and a second immunological reagent comprising a plurality of biotins;
(b) incubating the analyte sandwich with a molar excess, relative to the number of biotins on the analyte sandwich, of:
a linking member selected from avidin, or streptavidin, said linking member being labeled with a reporter group; and
a bridging member, each copy of said bridging member comprising an immunological reagent having no substantial affinity for said labeled linking member and no substantial affinity for said analyte sandwich, said bridging member further comprising a plurality of biotins,
thereby converting said analyte sandwich to a complex that includes a plurality of bound linking and bridging members, wherein:
at least one linking member is bound to both (i) a biotin of the second immunological reagent and (ii) a biotin of a first bound bridging member; and
at least one linking member is bound to both (i) a biotin of the first bound bridging member and (ii) a biotin of a second bound bridging member; and
(c) detecting signals from the reporter groups bound to said linking members of the complex as an indication of the presence of said analyte in said sample.

32. The method of claim 31 wherein said bridging member is a further copy of said second immunological reagent comprising a plurality of biotins.

33. The method of claim 31 wherein said linking member labeled with a reporter group is streptavidin labeled with a reporter group.

34. The method of claim 31 wherein said reporter group is phycoerythrin.

35. The method of claim 31 wherein said first immunological reagent is an antibody.

36. The method of claim 31 wherein said second immunological reagent is an antibody comprising a plurality of biotins.

37. The method of claim 31 wherein said bridging member is an antibody comprising a plurality of biotins.

38. The method of claim 31 wherein said first immunological reagent is an antibody fragment.

39. The method of claim 31 wherein said second immunological reagent is an antibody fragment comprising a plurality of biotins.

40. The method of claim 31 wherein said bridging member is an antibody fragment comprising a plurality of biotins.

41. The method of claim 31 wherein said complex formed in (b) further includes at least one labeled linking member bound to both (i) a biotin of the second bridging member and (ii) a biotin of a third bridging member.
